(19) United States
(12) Patent Application Publication
(10) Pub. No.: US 2007/0154497 A1
(43) Pub. Date: Jul. 5, 2007

(54) Stable, palatable syrup containing ibuprofen and method of its preparation

(76) Inventors: Michal Svoboda, Praha (CZ); Xenia Svobodova, Praha (CZ)

Correspondence Address: NOTARO AND MICHALOS, 100 DUTCH HILL ROAD, SUITE 110, ORANGEBURG, NY, 10962-2100, US

(21) Appl. No.: 10/588,870

(22) PCT Filed: Feb. 15, 2005
(86) PCT No.: PCT/CZ05/00019

§ 371 Date: Aug. 10, 2006

(30) Foreign Application Priority Data
Feb. 20, 2004 (CZ) PV 2004-262

(51) Int. Cl. A61K 31/724; A61K 31/192; A61K 9/00

(52) U.S. Cl. 424/400; 514/569; 514/58

(57) ABSTRACT

The invention concerns stable, palatable syrups for oral administration, containing S(+)-ibuprofen, hydroxypropyl beta-cyclodextrin, at least one sweetener and water, optionally essential oils. It also includes methods of preparation of the syrups of the invention, wherein crystalline S(+)-ibuprofen is dissolved in a hydroxypropyl beta-cyclodextrin aqueous solution and the final concentration of S(+)-ibuprofen is adjusted as desired by addition of an aqueous solution of sweeteners or a mixture of sweeteners and optionally of water. If the syrup of the invention contains essential oils, it is prepared by dissolving of crystalline S(+)-ibuprofen in a hydroxypropyl beta-cyclodextrin aqueous solution, combining the resulting solution with an essential oil solution in sweetener/mixture of sweeteners, preferably in a mixture of glycerin and 70% sorbitol aqueous solution, and adjusting the final concentration as desired by addition of aqueous solution of sweeteners or mixture of sweeteners and optionally of water.

### TECHNICAL FIELD

This invention relates to stable palatable syrups containing ibuprofen and method of their preparation. Such composition is formulated as orally administrable clear solution.

### BACKGROUND ART

Ibuprofen, the systematic chemical name of which is 2-(4-isobutylphenyl)-propionic acid, is a well known medicament having inflammatory, antipyretic and analgesic activities. Ibuprofen contains a single chiral centre at an asymmetrically substituted carbon atom, and therefore exists in two enantiomeric forms as S(+)-2-(4-isobutylphenyl)-propionic acid or as R(−)-2-(4-isobutylphenyl)-propionic acid. Although for many years ibuprofen has been used in therapy in the racemic form, it is already known that the active enantiomer is the S-enantiomer, further referred to as S(+)-ibuprofen. It is also known that in the absence of the R(−)-form the S(+)-ibuprofen has a substantially greater pharmacological potential than it was anticipated on the basis of comparison with the racemate activity, in particular the pure S(+)-ibuprofen acts faster.

Liquid compositions for oral administration, containing racemic ibuprofen, are known in the art. The problem of insolubility of ibuprofen in water is commonly solved by formulating the composition as suspension, containing adjuvants intended for masking the (almost outrageously) bitter taste of ibuprofen. When the suspension is administered, ibuprofen particles remaining in the mouth cause long-lasting unpleasant gustatory sensation. Another approach is to convert ibuprofen into a more soluble salt by treatment with a suitable base.

One such composition is described in U.S. Pat. No. 4,684,666 as a stable syrup suitable for oral administration, containing racemic ibuprofen in the amounts from 50 to 400 mg per 5 ml of syrup. Such composition has pH higher than 7.0 and lower than 7.7. Another such composition is described in U.S. Pat. No. 4,788,220 wherein the ibuprofen is maintained in suspension by suspending agents being xanthan gum, microcrystalline cellulose, sodium carboxymethyl cellulose and polysorbate 80. The unpleasant taste of ibuprofen is suppressed with sucrose and sorbitol solution, pH is within the range from 3.5 to 5.

Another possibility is the formation of an insoluble salt of ibuprofen, for example the aluminium salt. This possibility is described in U.S. Pat. No. 4,361,580. The aluminium salts of ibuprofen are essentially tasteless, insoluble in water, and are also formulated with suspending agents and sweeteners.

The ability of drug-cyclodextrin complexes to enhance the water solubility and to mask unpleasant taste and odour has been known for many years. For example U.S. Pat. No. 4,727,064 describes pharmaceutical preparations containing a drug with a substantially low water solubility and an amorphous water-soluble cyclodextrin mixture. Such preparations are called inclusion complexes and result in an improved solubility of the drug and more efficient absorption of the drug by the body. European patent EP 274444 describes the preparation of ibuprofen-cyclodextrin complexes using alpha-cyclodextrin, gamma-cyclodextrin or methylated beta-cyclodextrin instead of beta-cyclodextrin. On the contrary, U.S. Pat. No. 5,597,583 describes the use of ibuprofen-beta-cyclodextrin complex. British patent GB 2,219,585 describes complexes of beta-cyclodextrin with sodium, potassium, ammonium, magnesium or calcium salts of ibuprofen, or with ibuprofen salts of amino acids arginin, glycine or lysine. However, these complexes are not suitable for the preparation of formulations intended for oral administration in liquid dosage form, since, if dissolved in water, they confer an unpleasant, soapy taste, typical for an alkaline solution.

Use of hydroxypropyl beta-cyclodextrin for the preparation of palatable ibuprofen solutions is the subject of patent U.S. Pat. No. 5,024,997. The hydroxypropyl beta-cyclodextrin has a degree of hydroxypropyl substitution of about 6 to about 7.5. The weight ratio of ibuprofen to hydroxypropyl beta-cyclodextrin ranges from 1:11 to 1:15. Such orally administrable composition can additionally contain other cough or cold medicinal agents including pseudoephedrine hydrochloride, dextromethorphan hydrobromide and diphenhydramine hydrochloride.

All recited patents relate to racemic ibuprofen. Since the racemic ibuprofen has half the activity of S(+)-ibuprofen, the pharmaceutical preparations containing racemic ibuprofen must contain a 50% higher amount of the active ingredient to achieve the desired therapeutic effect, thus the organism is unnecessarily stressed with ballast substances.

Processes used for the racemic ibuprofen are not directly transferable to S(+)-ibuprofen, mainly because S(+)-ibuprofen has a substantially lower melting point (50 to 54° C.) compared to the racemate (75 to 78° C.), different physical properties such as a different behaviour when being dissolved in common solvents, and because under neutral and particularly under basic conditions the racemization of S(+)-ibuprofen occurs. Heating of the redundant R(−)-ibuprofen under basic conditions was published as the method of racemization yielding racemic mixture of R(−)- and S(+)-ibuprofen. Racemic ibuprofen is subsequently worked-up together with a new portion of racemic material by crystallization with a chiral amine into S(+)- and R(−)-ibuprofen. By this process, an optimal exploitation of the racemic material is achieved.

Therefore, compositions and processes leading to the preparation of stable liquid dosage pharmaceutical preparations containing racemic ibuprofen, for example that of U.S. Pat. No. 5,024,997, do not result in case of S(+)-ibuprofen in stable products, because of e.g. the formation of S(+)-ibuprofen microemulsion, slow racemization, etc.

The present invention brings a solution to the disadvantages of the formulations known in the art by finding a suitable composition of liquid dosage pharmaceutical preparation containing S(+)-ibuprofen and methods of preparation of such pharmaceutical preparation.

### DISCLOSURE OF INVENTION

The object of the present invention is a stable palatable syrup containing 0.01 to 2% (w/v) of S(+)-ibuprofen, preferably 1% of S(+)-ibuprofen, hydroxypropyl beta-cyclodextrin, at least one sweetener and water, optionally essential oils, wherein the weight ratio of S(+)-ibuprofen to hydroxypropyl beta-cyclodextrin is 1:10 to 1:18, preferably 1:10.8 to 1:12.

Sweeteners suitable in the palatable syrup of the present invention include sucrose, sorbitol solution, glycerine and the like, or their mixtures. The sweeteners are used to mask the acidic or sour taste of ibuprofen, which is typical for the organic acids, as distinguished from the normal (almost outrageously) bitter taste of ibuprofen, since this bitter taste is eliminated by the inclusion complex formed by the S(+)-ibuprofen with the hydroxypropyl beta-cyclodextrin.

The palatable syrup of the invention can further contain an optional amount of essential oils for taste correction or to achieve a further therapeutical effect. The essential oils to be used include orange, lemon, lemongrass or peppermint essential oil.

If desired, preservatives, colouring and flavouring agents and the like can be added as will be understood by those skilled in the art.

In the preferred embodiment, the pharmaceutical composition of the invention contains 50 mg of S(+)-ibuprofen per teaspoon (5 ml) of preparation.

Another aspect of this invention is a method of preparation of the palatable syrup of the invention, wherein crystalline S(+)-ibuprofen is dissolved at a temperature within the range from 15 to 50° C. in a 29-43% (w/w) hydroxypropyl beta-cyclodextrin aqueous solution and the final S(+)-ibuprofen concentration is adjusted as desired by addition of an aqueous solution of sweeteners and/or mixture of sweeteners and optionally of water.

In the preferred embodiment the concentration of hydroxypropyl beta-cyclodextrin aqueous solution is 31 to 34% (w/w). In another preferred embodiment the S(+)-ibuprofen is dissolved at a temperature within the range from 40 to 45° C.

A further aspect of this invention is a method of preparation of the palatable syrup of the invention, wherein crystalline S(+)-ibuprofen is dissolved at a temperature within the range from 15 to 50° C. in a 29-43% (w/w) hydroxypropyl beta-cyclodextrin aqueous solution, the resulting solution is combined with a solution of essential oil in suitable sweetener or mixture of sweeteners, preferably in a mixture of glycerine and 70% sorbitol aqueous solution, and the final S(+)-ibuprofen concentration is adjusted as desired by addition of aqueous solution of sweetener and/or mixture of sweeteners and optionally of water.

In the preferred embodiment the concentration of the hydroxypropyl beta-cyclodextrin aqueous solution is 31 to 34% (w/w). In another preferred embodiment the dissolution is performed at a temperature within the range from 40 to 45° C. Further embodiment of this preparation method is the addition of essential oils in the form of clear solution.

This invention is further illustrated by the following examples, which should not be construed as further limiting.

### EXAMPLES OF CARRYING OUT THE INVENTION

### Example 1

| Ingredient | % w/v | g/150 ml |
|---|---|---|
| Solution I | | |
| Purified water deionized | 24.0 | 36.0 |
| Hydroxypropyl beta-cyclodextrin | 10.8 | 16.2 |
| S (+)-ibuprofen | 1.0 | 1.5 |
| Solution II | | |
| Purified water deionized | 19.0 | 28.5 |
| Sucrose | 38.0 | 57.0 |
| Solution III | | |
| Glycerin | 5.0 | 7.5 |
| Sorbitol solution | 10.0 | 15.0 |
| Lemon essential oil | 0.17 | 0.26 |
| Purified water | q.s to | q.s |
| deionized to | 100 ml | 150 ml |

36 g of water was weighed into a first beaker, the beaker was placed into water bath. The hydroxypropyl beta-cyclodextrin was added gradually to the heated water and dissolved therein. The solution was heated to 40° C. The mixture was stirred until the solution was clear (ca 10 min). The S(+)-ibuprofen was added while stirring. Stirring was continued until the whole S(+)ibuprofen was dissolved, while the temperature was maintained at 40 to 45° C. The mixture was then cooled to 25° C. while stirring.

Into a second beaker fitted with water bath 28.5 g of water was weighed and heated to 70° C. while stirring. To the hot water the sucrose was added and left to dissolve. The mixture was then cooled to 25° C. while stirring.

Into a third container glycerine, sorbitol solution and lemon essential oil were weighed and stirred for 15 min. Subsequently, the S(+)-ibuprofen/cyclodextrin solution I and the sucrose solution II were added. The mixture was stirred for 10 min, water was then added to adjust the volume to 150 ml, and the resulting mixture was stirred for additional 3 hours.

The resulting syrup was clear, slightly acidic, with a pleasant citrus taste. There was no characteristic unpleasant ibuprofen taste.

The molar ratio of S(+)-ibuprofen to hydroxypropyl beta-cyclodextrin was 1:1.45, the weight ratio was 1:10.8.

### Example 2

| Ingredient | % w/v | g/150 ml |
|---|---|---|
| Solution I | | |
| Purified water deionized | 24.0 | 36.0 |
| Hydroxypropyl beta-cyclodextrin | 10.8 | 16.2 |
| S (+)-ibuprofen | 1.0 | 1.5 |
| Solution II | | |
| Purified water deionized | 19.0 | 28.5 |
| Sucrose | 19.0 | 28.5 |
| Solution III | | |
| Glycerine | 5.0 | 7.5 |
| Sorbitol solution | 5.0 | 7.5 |
| Lemon essential oil | 0.17 | 0.26 |
| Purified water deionized | q.s to | q.s to |
| | 100 ml | 150 ml |

The procedure for preparation of the pharmaceutical composition was the same as in Example 1. The resulting syrup was clear, slightly acidic and slightly bitter with lemon taste and odour.

It has been found that:

a) when using a weight ratio of S(+)-ibuprofen to hydroxypropyl beta-cyclodextrin less than 1:10 the S(+)-ibuprofen is not dissolved totally;
b) a weight ratio of S(+)-ibuprofen to hydroxypropyl beta-cyclodextrin more than 1:18 is too high and is uneconomical.
   S(+)-ibuprofen syrup stability assay was carried out as follows:

The stability of S(+)-ibuprofen content in syrups, prepared in analogous way as in Examples 1 and 2 with lemon, orange and peppermint flavour, was tested under following conditions: 50° C., 50° C. alternate with refrigerator temperature and 50° C. alternate with room temperature. Furthermore, the effect of light was investigated by maintaining the syrups at room temperature/light or room temperature/dark.

The following results were obtained:

The results are summarised in tables I-IV. The values of S(+)-ibuprofen content are given in %. Only results for syrup with lemon taste are shown, the results for syrups with orange and peppermint taste were similar.

TABLE I
Temperature 50° C.

| Start | 3 days | 7 days | 14 days |
|---|---|---|---|
| 0.76 | 0.76 | 0.77 | 0.73 |

TABLE II
Alternate regime 50° C./Refrigerator

| Start | 3 days | 7 days | 14 days |
|---|---|---|---|
| 0.76 | 0.76 | 0.75 | 0.76 |

TABLE III
Alternate regime 50° C./Room temperature

| Start | 3 days | 7 days | 14 days |
|---|---|---|---|
| 0.76 | 0.77 | 0.75 | 0.73 |

TABLE IV
Room temperature/light, dark

| Start | light | dark |
|---|---|---|
| 0.76 | 0.75 | 0.76 |

Enantiomeric purity of the prepared syrups after the stability test was investigated as well. The content of R-enantiomer remained in all cases changeless at the level of the input value, about 0.2%.

After data evaluation it has been found that the S(+)-ibuprofen containing syrup of the present invention is stable for 14 days when stored at elevated temperature, at room temperature, or in refrigerator, i.e. at temperatures ranging from 5 to 50° C. Under these conditions neither changes in the S(+)-ibuprofen content occur nor is the S(+)-ibuprofen converted into R(−)-enantiomer.

1. Stable, palatable syrup containing S(+)-ibuprofen, characterised in that it contains 0.01 to 2% (w/v) of S(+)-ibuprofen, preferably 1% of S(+)-ibuprofen, hydroxypropyl beta-cyclodextrin, at least one sweetener, water, and optionally essential oils, wherein the weight ratio of S(+)-ibuprofen to hydroxypropyl beta-cyclodextrin is 1:10 to 1: 18, preferably 1:10.8 to 1:12.

2. The syrup according to claim 1, characterised in that the sweetener is selected from the group consisting of sucrose, sorbitol and glycerin, and preferably their combination.

3. The syrup according to claim 1, characterised in that it contains one or more sweeteners selected from the group consisting of sucrose, sorbitol and glycerin, and preferably their combination, and one or more essential oils selected from the group consisting of lemon, orange, peppermint and lemongrass essential oils.

4. A method of preparation of the syrup of claim 1, characterised in that crystalline S(+)-ibuprofen is dissolved at a temperature within the range from 15 to 50° C. in a 29-43% (w/w) hydroxypropyl beta-cyclodextrin aqueous solution and the final S(+)-ibuprofen concentration is adjusted as desired by addition of aqueous solution of sweeteners and/or mixture of sweeteners and optionally of water.

5. The method according to claim 4, characterised in that the concentration of hydroxypropyl beta-cyclodextrin aqueous solution is 31 to 34% (w/w).

6. The method according to claim 4, characterised in that the S(+)-ibuprofen is dissolved at temperature within the range from 40 to 45° C.

7. A method of preparation of the syrup of claim 3, characterised in that crystalline S(+)-ibuprofen is dissolved at a temperature within the range from 15 to 50° C. in a 29-43% (w/w) hydroxypropyl beta-cyclodextrin aqueous solution, the resulting solution is combined with a solution of an essential oil in a suitable sweetener or mixture of sweeteners, preferably in a mixture of glycerin and 70% sorbitol aqueous solution, and the final S(+)-ibuprofen concentration is adjusted as desired by addition of an aqueous solution of sweetener and/or mixture of sweeteners and optionally of water.

8. The method according to claim 7, characterised in that the concentration of hydroxypropyl beta-cyclodextrin aqueous solution is 31 to 34% (w/w).

9. The method according to claim 7, characterised in that the S(+)-ibuprofen is dissolved at temperature within the range from 15 to 50° C., preferably within the range from 40 to 45° C.

10. The method according to claim 7, characterised in that the essential oils are added in the form of a clear solution.
